(19) United States
(12) Patent Application Publication
(10) Pub. No.: US 2010/0075922 A1
(43) Pub. Date: Mar. 25, 2010

(54) HEPARINS INCLUDING AT LEAST ONE COVALENT BOND WITH BIOTIN OR A BIOTIN DERIVATIVE, METHOD FOR PREPARING SAME AND USE THEREOF

(75) Inventors: Christian VISKOV, Ris Orangis (FR); Philippe HUBERT, Maisons-Alfort (FR); Pierre MOURIER, Charenton LePont (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/539,237

(22) Filed: Aug. 11, 2009

Related U.S. Application Data
(63) Continuation of application No. PCT/FR2008/000172, filed on Feb. 12, 2008.

(30) Foreign Application Priority Data
Feb. 14, 2007 (FR) 0701053

(51) Int. Cl. A61K 31/727; C08B 37/10; C12P 19/26; A61P 9/00

(52) U.S. Cl. 514/56; 536/21; 435/84

(57) ABSTRACT

The invention relates to biotinylated heparins comprising constituent polysaccharides having at their reducing ends at least one covalent bond with biotin or a biotin derivative, and also to the process for preparing them, to pharmaceutical compositions containing them and to their therapeutic use. The biotinylated polysaccharides are of the formula (I)

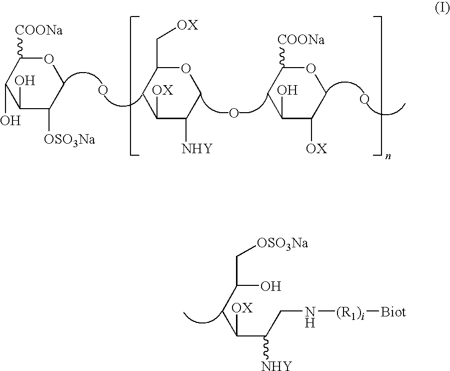

with the variables as defined herein.

### FIELD OF THE INVENTION

The present invention relates to heparins containing at least one covalent bond with biotin or a biotin derivative, and also to the process for preparing them, to pharmaceutical compositions containing them and to their therapeutic use.

### BACKGROUND OF THE INVENTION

Heparin is a mixture of sulfated mucopolysaccharides of animal origin, with a molecular weight in the region of 15 000 daltons (Da). Reference will be made hereinbelow to heparin or heparins: the structures, average molecular masses and polydispersity of the polysaccharide chains of heparin may in fact vary according to the animal species and the organ from which the heparin is derived (examples: porcine mucous heparin, bovine intestinal heparin, etc.).

Heparin catalyses, especially via antithrombin III (ATIII), the inhibition of two enzymes that participate in the blood coagulation cascade, namely factor Xa and factor 11a (or thrombin). It is thus used therapeutically for its anticoagulant and antithrombotic properties.

However, heparin has drawbacks that limit its conditions of use. In particular, its high anticoagulant activity (especially its high anti-factor IIa activity) may cause hemorrhaging (Seminars in Thrombosis and Hemostasis, vol. 5, sup. 3, 1999). Heparin derivatives are known for these undesirable hemorrhagic side effects.

In the field of treating thrombosis, the aim is to reestablish or maintain blood fluidity while at the same time avoiding the induction of a hemorrhage. In point of fact, it is well known that, for any accidental reason, a hemorrhage may be triggered in a patient under treatment. There may also be a need to perform a surgical operation on a patient under antithrombotic treatment. Furthermore, in the course of certain surgical operations, anticoagulants may be used at high dose so as to prevent coagulation of the blood, and it is desirable to neutralize them at the end of the operation. There is thus a need for neutralizable antithrombotic agents to stop the anticoagulant activity at any moment.

Neutralizable antithrombotic agents, such as biotinylated synthetic polysaccharides, have been described in patent applications WO 02/24754 and WO 06/030104. Their synthesis, especially comprising the grafting of biotin or of the biotin derivative performed on protected equivalents of the polysaccharides mentioned above rather than on these polysaccharides themselves, is not applicable to the compounds of the present invention. The reason for this is that it is desired to perform the biotinylation on finished products, which are mixtures of polysaccharides and are thus heterogeneous products, on which the grafting of biotin as described in the abovementioned patent applications would not make it possible to induce a sufficient regioselectivity of the grafting position and would not allow biotinylation of all the functionalizable polysaccharide chains of heparins.

The team of Osmond et al. describes, in Analytical Biochemistry, 31 (2002) 199-207, several techniques for biotinylating a porcine heparin, one of them being described as involving a coupling of biotin at the reducing end of a heparin via a reductive amination followed by coupling with biotin. However, the operating conditions described in the said document do not allow biotinylated heparins to be obtained fully and reproducibly: they do not take into account the structural diversity of heparins and the real structure of the polysaccharide chains as present in the commercially available heparins. The latter heparins comprise a large proportion of polysaccharide chains that contain at their reducing end a degraded glycoserine, which is not functionalizable with biotin according to the protocol described by Osmond et al. Thus, the operating conditions described in the said publication for the biotinylation of porcine heparin do not allow biotinylated heparins to be obtained fully and reproducibly with expected characteristics, such as a degree of biotinylation sufficient to allow efficient neutralization.

The team of Tseng et al. describes, in Biomaterials, 27 (2006), 2627-2636, a technique for immobilizing heparin on films by interaction with avidin, following functionalization of the heparin with biotin. The biotinylation of heparin is performed via oxidation with iodine, followed by the formation of a lactone, and then coupling with a biotin 2-(4-aminophenyl)ethylamine derivative. The operating conditions presented by Tseng et al. do not, however, assume the total and reproducible production of heparins biotinylated at the reducing end: specifically, there is nothing to indicate that the oxidation step may be selective on the reducing end, or that the biological activity of heparin is conserved after such a treatment.

The team of Kett et al. describes, in Biochimica and Biophysica Acta, 1620 (2003), 225-234, affinity studies between avidin and various glycosaminoglycans, and demonstrates that avidin shows strong affinity for heparin. In these affinity studies, heparin may be derivatized with biotin, but there is no indication of the site of biotinylation in the polysaccharide chains of heparin.

The Applicant thus set itself the aim of providing novel heparins that can be neutralized with avidin or streptavidin and that have biological properties comparable to the native heparins.

### SUMMARY OF THE INVENTION

The present invention relates to novel modified heparins, referred to hereinbelow as “biotinylated heparins”, characterized in that the constituent polysaccharides have at their reducing end a covalent bond to a group-(R1)i-Biot and correspond to the general formula (I):

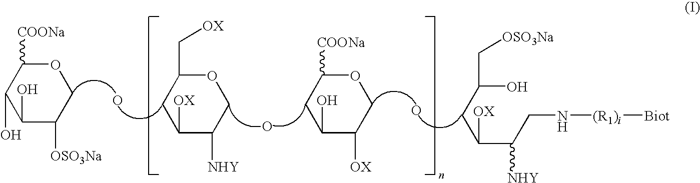

in which:
i is equal to 0 or 1,
R1 represents a sequence of formula (a) or (b):

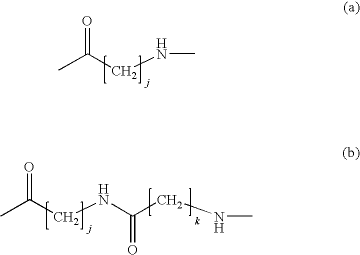

in which j and k, which may be identical or different, are integers that may take any value from 1 to 10,
Biot represents a biotin group or biotin derivative,
n represents an integer with a mean value of about 25 for a heparin with an average molecular mass of 15 000 Da,
X represents H or SO₃Na,
Y represents COCH₃ or SO₃Na,
the wavy line denotes a bond located either below or above the plane of the pyranose ring to which it is attached,
   and also the pharmaceutically acceptable salts thereof.

### DETAILED DESCRIPTION OF THE INVENTION

Surprisingly, the introduction of biotin or of a biotin derivative at the reducing end of the polysaccharide chains does not modify the pharmacological activity of the heparins. Specifically, the novel biotinylated heparins that are the subject of the invention have antithrombotic activities comparable to native heparins, i.e. heparins before biotinylation.

They have a considerable advantage over native heparins: they may be rapidly neutralized with a specific antidote, in the case of emergency. This specific antidote is avidin, in tetrameric or monomeric form, or streptavidin, with respective masses equal to about 66 000, 16 400 and 60 000 Da (The Merck Index, Twelfth edition, 1996, M. N. 920, pages 151-152, Revue Pierce Avidin-Biotin Handbook).

They also have the advantage of being useful in therapeutic indications for which the doses used are higher, while at the same time reducing the risk of hemorrhage; they may thus be useful in the arterial therapeutic field.

The biotin (Biot) group mentioned above is a radical derived from hexahydro-2-oxo-1H-thieno[3,4-d]imidazole-4-pentanoic acid. Advantageously, the Biot group in the general formula (I) according to the invention corresponds to formula (c):

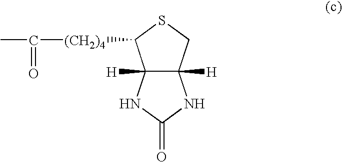

The biotin derivatives are commercially available (“Pierce” Biotin-avidin products catalogue, 2005, pp. 7-11) or may be prepared using standard methods known to those skilled in the art. Mention may be made especially of the biotin derivatives mentioned in patent application WO 02/24754.

In the biotinylated heparins according to the invention, the index i may be equal to 0, in which case the bond with biotin or the biotin derivative is made directly on the amine function borne by the saccharide unit on the reducing end of the polysaccharide chains.

Alternatively, i may be equal to 1 and the bond with the biotin group or biotin derivative may consist, for example, of a sequence of formula (a) above in which j is equal to 5, or of a sequence of formula (b) above in which j and k are identical and are equal to 5. Thus, in formula (I) above, R1 may represent, for example, a sequence of formula —CO—(CH₂)₅—NH or —CO—(CH₂)₅—NH—CO—(CH₂)₅—NH—.

In the context of the present invention, the term “reducing end” means the end of the polysaccharide chain in which the terminal glucosamine or mannosamine (mannosamine resulting from an epimerization in basic medium of glucosamine) has a cyclic hemiacetal function, corresponding to formula (II) below:

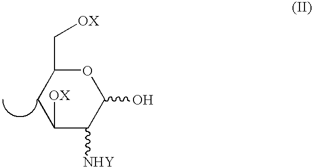

in which
X represents H or SO₃Na,
Y represents COCH₃ or SO₃Na, and
the wavy line denotes a bond located either below or above the plane of the pyranose ring to which it is attached (below: glucosamine, above: mannosamine).

The term “constituent polysaccharides of heparin” means polysaccharides characterized by the repetition of a disaccharide unit containing a uronic acid residue (D-glucuronic acid or L-iduronic acid) and a D-glucosamine residue, which may be N-sulfated or N-acetylated. The disaccharide unit may also be O-sulfated in positions C6 and/or C3 of D-glucosamine and in position C2 of uronic acid (Heparin-binding proteins, H. Edward Conrad, 1998, p. 1).

As indicated previously, heparin is a mixture of sulfated mucopolysaccharides of animal origin. Native heparins, i.e. the starting heparins before biotinylation, are referred to as “heparins”.

The heparins used in the present invention may especially be of bovine, ovine or porcine origin; more specifically, they may be derived from bovine lungs, from bovine intestinal mucosae, from pig intestinal mucosae or from sheep intestinal mucosae. Advantageously, the heparins used in the present invention are of porcine origin, for example derived from pig intestinal mucosae.

The biotinylated heparins according to the present invention are such that at least 60%, advantageously at least 64%, of the constituent polysaccharides of the said heparins have at their reducing end a covalent bond to a group —(R1)ᵢ-Biot and correspond to formula (I) as defined above, irrespective of the original structure of the heparin; advantageously, at least 80% of the constituent polysaccharides of the said heparins have at their reducing end a covalent bond to a group —(R1)ᵢ-Biot.

The invention covers biotinylated heparins in the form of any of their pharmaceutically acceptable salts.

A subject of the present invention is also a process for preparing the biotinylated heparins mentioned above, characterized in that:

a) heparin is treated with heparinase 3,
b) a reductive amination is then performed, on the product obtained above, in the presence of an amine salt and a reducing agent, at a temperature of between 20 and 80° C.,
c) an acylation is lastly performed with an activated group —(R1)ᵢ-Biot, in which R1, i and Biot are as defined in relation with formula (I) above, in the presence of a base in aqueous medium or in organic medium.

The steps of the above preparation process may be controlled by analytical HPLC monitoring, especially of SAX type, and more particularly of CTA-SAX type, after depolymerization of the constituents of the heparin-based mixture in the presence of a mixture of heparinases 1, 2 and 3. Such analytical monitoring may be performed, for example, using the method described in patent application US 2005/0186679 A1.

It is especially confirmed, after the reductive amination step b), that at least 80%, advantageously at least 90%, of the constituent polysaccharides of the said heparins bear at their reducing end an —NH₂ function (amino-reduced polysaccharides).

It is especially confirmed, after the acylation step c), that at least 80%, advantageously at least 90%, of the said amino-reduced polysaccharides are biotinylated.

The overall yield of the process for preparing the biotinylated heparins according to the invention is thus at least 60% and advantageously at least 64%. Advantageously, this yield is at least 80%.

The terms below are defined as follows:
heparinase 1: the enzyme heparin lyase I (EC 4.2.2.7) from Flavobacterium heparinum,
heparinase 2: the enzyme heparin lyase II from Flavobacterium heparinum,
heparinase 3: the enzyme heparin lyase III (EC 4.2.2.8) from Flavobacterium heparinum.

Heparinase 3 enables removal of the region for binding to proteins (glycoserine) of the polysaccharide chains of the heparins and allows the production of chains whose reducing ends are free of glycoserine residues. Heparinases 1 and 2 enable cleavage of the polysaccharide chains to fragments of low molecular weight (heparin depolymerization reactions).

The process for preparing the compounds according to the invention uses as starting heparins (“native” heparins) heparins prepared as reported previously in the literature. Reference will be made especially to the publication “L'héparine, fabrication, structure, propriétés, analyses”, J. P. Duclos, published by Masson, 1984. The heparins may especially be prepared according to the process described in patent application US 2005/0215519 A1.

In the reductive amination step b) of the above preparation process, the amine salt may be a quaternary amine salt; it is advantageously an ammonium halide salt corresponding to the formula NH₄Z, in which Z represents a halogen atom, such as a chlorine, fluorine, bromine or iodine atom.

In the reductive amination step b) of the above preparation process, the reducing agent may be a borohydride salt, for example a cyanoborohydride salt.

In the reductive amination step b) of the above preparation process, the temperature is advantageously between 50 and 80° C.

In the acylation step c) of the above preparation process, the base may be a carbonate or hydrogen carbonate salt, especially in sodium or potassium salt form, or alternatively any water-soluble or organo-soluble organic base known to those skilled in the art.

In the acylation step c) of the above preparation process, the term “organic medium” means, for example, dichloromethane or dimethylformamide.

The process for preparing the biotinylated heparins according to the invention advantageously comprises the following steps:

a) heparin is treated with heparinase 3,
b) a reductive amination is performed on the product obtained above in the presence of an ammonium halide salt and a borohydride salt, at a temperature of between 50 and 80° C.,
c) lastly an acylation is performed with a group —(R1)ᵢ-Biot as defined above in activated ester form, in the presence of a base in aqueous medium.

The biotinylated derivatives —(R1)ᵢ-Biot as defined above may be used in the acylation reaction directly in the form of activated esters, preformed or generated in situ using standard coupling conditions known to those skilled in the art. Activated esters in the form of N-hydroxysuccinimide derivatives or of 3-sulfo-N-hydroxy-succinimide derivatives may especially be used.

The preparation process according to the invention is illustrated in Scheme 1.

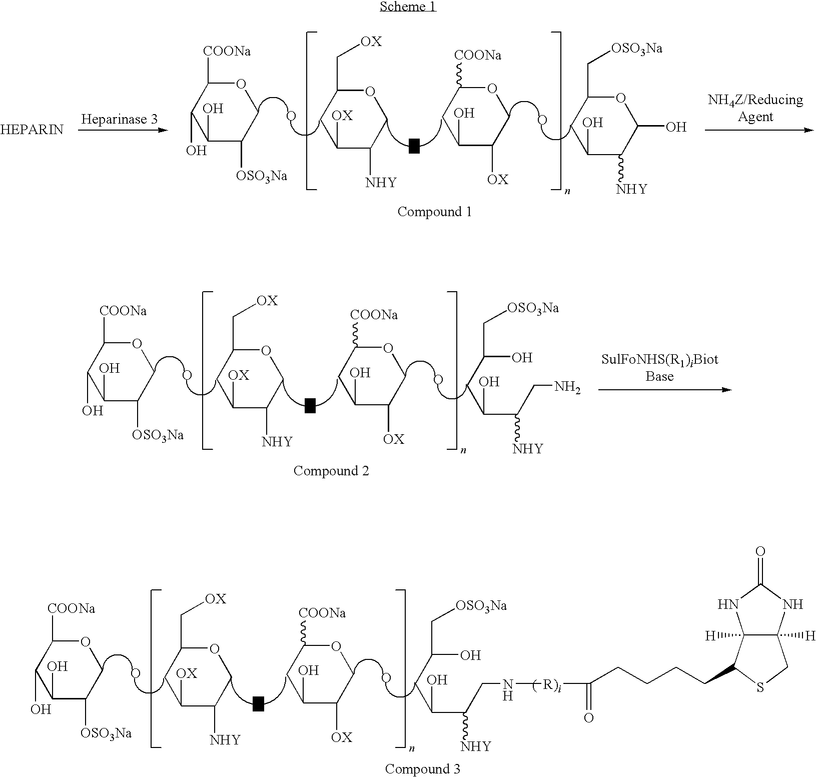

According to Scheme 1, the heparin is treated with heparinase 3 to remove the remaining protein bond (i.e. the native or degraded glycoserines) and to obtain a compound 1 comprising a reducing end free of glycoserine residues. This heparin (compound 1) may then be subjected to a reductive amination to produce compound 2, containing a free amine function at the reducing end, in the presence of an amine salt and a reducing agent such as a borohydride salt.

This compound may then be acylated to provide the biotinylated compound 3, via reaction with an activated biotin derivative —(R1)ᵢ-Biot, as defined above, in the presence of a base. This reaction may be performed, for example, with the sodium salt of the ester 3-sulfosuccinimidyl 6-biotinamidohexanoyl hexanoate when R1 represents the sequence —CO—(CH₂)₅—NH—CO—(CH₂)₅—NH—, or with the sodium salt of the ester 3-sulfosuccinimidyl 6-biotinamido hexanoate when R1 represents the sequence —CO—(CH₂)₅—NH—, or alternatively with the sodium salt of the biotinoyl-3-sulfosuccinimidyl ester when R1 is not present (i=0).

In Scheme 1, it is understood that the compounds 1, 2 and 3 are a theoretical representation, since it is a matter in reality, as heparin derivatives of mixtures of polysaccharide chains.

In the text hereinbelow, examples of synthesis of the biotinylated heparins according to the invention and of various intermediates that are useful for obtaining them are detailed by way of illustration.

The following abbreviations are used:

EPB heparin: the heparin sold by the company Bioiberica;
HPLC: high-performance liquid chromatography;
SAX: strong anion exchange chromatography;
CTA: cetyl trimethyl ammonium;
qs: quantity sufficient;
LC: long chain, corresponding to the 6-aminohexanoyl sequence;
sulfo-NHS: sodium salt of the 3-sulfosuccinimidyl ester;
Heparinase 1: heparin lyase I enzyme (EC 4.2.2.7) from Flavobacterium heparinum
Heparinase 2: heparin lyase II enzyme from Flavobacterium heparinum;
Heparinase 3: heparin lyase III (EC 4.2.2.8) from Flavobacterium heparinum.

### Example 1

### 1.1 EPB Heparin Purified by Depolymerization with Heparinase 3

At a temperature in the region of 20° C., 1 g of raw EPB heparin is dissolved in 15 ml of aqueous 5 mM sodium phosphate solution adjusted to pH 7.0±0.1, 20 mM sodium chloride and 1 mg/ml of BSA. 0.5 IU of heparinase 3 is added to the heparin solution. The reaction mixture obtained is stirred for 5 days. 1 g of sodium chloride and 45 ml of methanol are added to the reaction mixture. The suspension obtained is filtered through a 0.45 μm membrane. The cake is washed with methanol and with diethyl ether, and then dried under vacuum. 0.98 g of a white solid is obtained. The observed yield is 98%.

The product may be controlled by depolymerization using a mixture of heparinases 1, 2 and 3 and analyzed by HPLC-SAX using the method described in patent application US 2005/0186679 A1. The results show a disappearance of at least 80% of the glycoserine species present in the starting heparin.

¹H NMR spectrum of the polysaccharide mixture in D₂O (25° C., δ in ppm), main signals: 2.05 (CH₃CO, s), 3.28 (CH, m), 3.65 (CH, m), 3.77 (CH, m), 4.05 (CH, m), 4.10 (CH, s), 4.20 (CH, s), 4.25 (CH, d, 12 Hz), 4.35 (CH, s), 4.42 (CH, m), 4.88 (CH, s), 5.22 (CH, s), 5.42 (CH, s).

### 1.2 Heparin Depolymerization with Amino-Reduced Heparinase 3

0.5 g of heparin purified with heparinase 3 is dissolved in aqueous 5 M ammonium chloride solution. 0.5 g of sodium cyanoborohydride is added to the heparin solution. The mixture is maintained at 70° C. for 24 hours. The solution is cooled to a temperature in the region of 20° C., diluted with water (qs 50 ml) and then desalified on a column of Sephadex G10. The fraction obtained is injected onto a Q-Sepharose column. The product is diluted with water and then with a gradient of sodium perchlorate. The product obtained is desalified on a column of Sephadex G10 and then freeze-dried. 444 mg of a white lyophilizate are obtained. The observed yield is 89%. The product is used without further purification in the following acylation step.

¹H NMR spectrum of the polysaccharide mixture in D₂O (25° C., δ in ppm), main signals: 2.05 (CH₃CO, s), 3.28 (CH, m), 3.65 (CH, m), 3.77 (CH, m), 4.05 (CH, m), 4.10 (CH, s), 4.20 (CH, s), 4.25 (CH, d, 12 Hz), 4.35 (CH, s), 4.42 (CH, m), 4.88 (CH, s), 5.22 (CH, s), 5.42 (CH, s).

### 1.3 Biotinylated and Amino-Reduced Heparin Depolymerized with Heparinase 3

200 mg of amino-reduced depolymerized heparin are dissolved in 2 ml of 0.5 M sodium hydrogen carbonate solution at a temperature in the region of 20° C. 37 mg of sulfo-NHS-LC-biotin are added to the solution obtained. The solution obtained is stirred at a temperature in the region of 20° C. for 1 hour. The solution is diluted with 4 ml of 0.5 M sodium hydrogen carbonate solution. 37 mg of sulfo-NHS-LC-biotin are added and the mixture obtained is stirred for 2 hours. A further 37 mg of sulfo-NHS-LC-biotin are added and the reaction mixture is stirred for 16 hours. The reaction medium obtained is diluted with water (qs 200 ml), filtered on a 0.45 μm membrane and injected onto a Q-Sepharose column. The product is eluted with water and then with a gradient of sodium perchlorate. The product obtained is desalified on a column of Sephadex G10 then freeze-dried. 188 mg of a white lyophilizate are obtained. The observed yield is 94%.

The product may be controlled by depolymerization using a mixture of heparinases 1, 2 and 3 and analyzed by HPLC-SAX, using the method described in patent application US 2005/0186679 A1.

¹H NMR spectrum of the polysaccharide mixture in D₂O (25° C., δ in ppm), main signals: between 1.3 and 1.8 (CH₂ biotin, m), 2.05 (CH₃CO, s), 2.25 (CH₂CO biotin, t, 7 Hz), 2.79 (1H, d, 12 Hz), 3.00 (1H, dd, 12 and 5 Hz), 3.20 (NCH₂ biotin, m), 3.30 (CH, m), 3.68 (CH, m), 3.80 (CH, m), 4.05 (CH, m), 4.10 (CH, s), 4.20 (CH, s), 4.25 (CH, m), 4.35 (CH, s), 4.45 (CH, m), 4.63 (NCH biotin, m), 4.85 (CH, s), 5.22 (CH, s), 5.42 (CH, s).

### Example 2

### 2.1 Heparin Obtained According to US 2005/0215519 A1, Depolymerization with Heparinase 3

At a temperature in the region of 20° C., 1 g of heparin according to US 2005/0215519 A1 is dissolved in 15 ml of aqueous 5 mM sodium phosphate solution adjusted to pH 7.0±0.1, 20 mM sodium chloride and 1 mg/ml of BSA. 0.5 IU of heparinase 3 is added to the heparin solution. The reaction mixture obtained is stirred for 7 days. 1 g of sodium chloride and 45 ml of methanol are added to the reaction mixture. The suspension obtained is filtered through a 0.45 μm membrane. The cake is washed with methanol and with diethyl ether, and then dried under vacuum. 0.96 g of a white solid is obtained. The observed yield is 96%.

The product may be controlled by depolymerization using a mixture of heparinases 1, 2 and 3 and analyzed by HPLC-SAX using the method described in patent application US 2005/0186679 A1. The results show an at least 80% disappearance of the glycoserine species present in the starting heparin.

¹H NMR spectrum of the polysaccharide mixture in D₂O (25° C., δ in ppm), main signals: 2.05 (CH₃CO, s), 3.28 (CH, m), 3.65 (CH, m), 3.77 (CH, m), 4.05 (CH, m), 4.10 (CH, s), 4.20 (CH, s), 4.25 (CH, d, 12 Hz), 4.35 (CH, s), 4.42 (CH, m), 4.88 (CH, s), 5.22 (CH, s), 5.42 (CH, s).

### 2.2 Heparin According to Us 2005/0215519 A1 Depolymerized with Heparinase 3 and Amino-Reduced

0.5 g of heparin according of US 2005/0215519 A1 treated with heparinase 3 are dissolved in 20 ml of aqueous 5 M ammonium chloride solution. 0.5 g of sodium cyanoborohydride are added to the heparin solution. The mixture is maintained at 70° C. for 24 hours. The solution is cooled to a temperature in the region of 20° C. and diluted with water (qs 50 ml), then desalified on a column of Sephadex G10. The fraction collected is freeze-dried. 446 mg of a white lyophilizate are obtained. The observed yield is 89%. The product is used without further purification in the following acylation step.

¹H NMR spectrum of the polysaccharide mixture in D₂O (25° C., δ in ppm), main signals: 2.05 (CH₃CO, s), 3.28 (CH, m), 3.65 (CH, m), 3.77 (CH, m), 4.05 (CH, m), 4.10 (CH, s), 4.20 (CH, s), 4.25 (CH, d, 12 Hz), 4.35 (CH, s), 4.42 (CH, m), 4.88 (CH, s), 5.22 (CH, s), 5.42 (CH, s).

### 2.3 Biotinylated and Amino-Reduced Heparin According to Us 2005/0215519 A1 Depolymerized with Heparinase 3

200 mg of amino-reduced depolymerized heparin are dissolved in 2 ml of 0.5 M sodium hydrogen carbonate solution at a temperature in the region of 20° C. 37 mg of sulfo-NHS-LC-biotin are added to the solution obtained. The solution is stirred at a temperature in the region 20° C. for 1 hour. The suspension obtained is diluted with 4 ml of 0.5 M sodium hydrogen carbonate solution. 37 mg of sulfo-NHS-LC-biotin are added and the mixture is stirred for 2 hours. A further 37 mg of sulfo-NHS-LC-biotin are added and the reaction mixture is stirred for 16 hours. The reaction medium obtained is diluted with water (qs 200 ml), filtered through a 0.45 μm membrane and injected onto a Q-Sepharose column. The product is eluted with water and then with a gradient of sodium perchlorate. The product obtained is desalified on a column of Sephadex G10, then freeze-dried. 188 mg of a white lyophilizate are obtained. The observed yield is 94%.

The product can be controlled by depolymerization using a mixture of heparinases 1, 2 and 3 and analyzed by HPLC-SAX using the method described in patent application US 2005/0186679 A1.

¹H NMR spectrum of the polysaccharide mixture in D₂O (25° C., δ in ppm), main signals: between 1.3 and 1.8 (CH₂ biotin, m), 2.05 (CH₃CO, s), 2.25 (CH₂C0 biotin, t, 7 Hz), 2.79 (1H, d, 12 Hz), 3.00 (1H, dd, 12 and 5 Hz), 3.20 (NCH₂ biotin, m), 3.30 (CH, m), 3.68 (CH, m), 3.80 (CH, m), 4.05 (CH, m), 4.10 (CH, s), 4.20 (CH, s), 4.25 (CH, m), 4.35 (CH, s), 4.45 (CH, m), 4.63 (NCH biotin, m), 4.85 (CH, s), 5.22 (CH, s), 5.42 (CH, s).

The compounds according to the invention were subjected to biochemical and pharmacological studies.

### Measurement of the Anti-Factor IIa Activity and of the Anti-Factor Xa Activity

The anti-factor IIa (anti-FIIa) activity and the anti-factor Xa (anti-FXa) activity in human plasma or a buffer system are analyzed via a chromogenic method: the anti-factor IIa activity is tested by means of the Actichrome heparin anti-factor IIa kit (American Diagnostica) containing the chromogenic substrate S-2238, α-thrombin and human ATIII (antithrombin III). The anti-FXa activity is determined with the automated coagulation instrument ACL 7000 (Instrumentation Laboratory) using the Heparin kit (Instrumentation Laboratory) containing ATIII, factor Xa and the chromogenic substrate S-2765. The two analyses are performed according to the manufacturer's instructions.

The following standards are used to establish a standard calibration curve for measuring the in vitro activity of the biotinylated heparin fractions in human plasma and the buffer system:
1st international standard for low molecular weight heparins (National Institute for Biological Standards and Control, London, UK, established in 1987, code No. 85/600),
2nd international standard for low molecular weight heparins (National Institute for Biological Standards and Control, London, UK, established in 1987, code No. 01/608, used since June 2006)
enoxaparin (Clexane®, sanofi-aventis, France) was used as internal reference.

For the determinations of anti-FIIa activity, 10 μl of sample or of international low molecular weight heparin standards are diluted to 1:16 with antithrombin in human plasma or the buffer system containing 0.05 M Tris HCl, 0.154 M NaCl, at pH 7.4. 10 μl of this solution are added to a 96-well microtitration plate. The measurement is repeated in triplicate (on 3 wells). The microtitration plate is maintained at 37° C. while agitating at 300 rpm. 40 μl of thrombin are added to each of the wells and incubated for exactly 2 minutes. 40 μl of Spectrozyme are added. After 90 seconds, the reaction is stopped by adding 40 μl of acetic acid. The absorption is measured at 405 nm using a SpectraMax 340 (Molecular Devices).

For the anti-FXa activity measurements, the sample or the international low molecular weight heparin standards are diluted in human plasma or the buffer system containing 0.05 M Tris HCl, 0.154 M NaCl, pH 7.4. The samples containing the heparinoids in the plasma or the buffer are again diluted to 1:20 with a working buffer containing ATIII, and placed in duplicate in the probe rotor. The factor Xa reagent and the chromogenic substrate are poured into the indicated reservoirs of the automated coagulation instrument ACL 7000.

The anti-FXa activity measurement is performed with the “heparin” protocol integrated into the ACL 7000 software. During the analysis, 50 μl of the sample (diluted with the working buffer) are mixed with 50 μl of the factor Xa reagent. After an incubation time of 60 seconds at 37° C., 50 μl of the chromogenic substrate of concentration 1.1 mM are added and the changes in absorption as a function of time are measured at a wavelength of 405 nm.

The results obtained are described especially in the table below.

| | Anti-FXa activity |
|---|---|
| | (IU/mg) |
| EPB heparin | 235 |
| EPB heparin purified with heparinase 3 | 251 |
| EPB heparin purified with heparinase 3 and amino- | 217 |
| reduced | |
| EPB heparin purified with heparinase 3, amino- | 218 |
| reduced and biotinylated | |
| Heparin according to US 2005/0215519 A1 | 214 |
| Heparin according to US 2005/0215519 A1 purified | 257 |
| with heparinase 3 | |
| Heparin according to US 2005/0215519 A1 purified | 203 |
| with heparinase 3 and amino-reduced | |
| Heparin according to US 2005/0215519 A1 purified | 214 |
| with heparinase 3, amino-reduced and biotinylated | |

It is thus seen that the anti-FXa activity of the biotinylated heparins according to the invention is conserved, by comparison with the starting native heparins.

The biotinylated heparins according to the present invention may be used for the preparation of medicaments. They may especially be used as antithrombotic medicaments. Thus, according to another of its aspects, a subject of the invention is medicaments comprising a biotinylated heparin as defined above. These medicaments find their use in therapeutics, in particular in the treatment and prevention of venous thrombosis, arterial thrombotic accidents, especially in the case of myocardial infarction or unstable angina, peripheral arterial thrombosis, such as arteriopathy of the lower limbs, cerebral arterial thrombosis and strokes. They are also useful in the prevention and treatment of the proliferation of smooth muscle cells, angiogenesis, and as neuroprotective agents for atherosclerosis and arteriosclerosis.

According to another of its aspects, the present invention also relates to a method for treating the abovementioned pathologies, which comprises the administration to a patient of an effective dose of a compound according to the invention, or of a pharmaceutically acceptable salt thereof. The use of the biotinylated heparins as defined above for treating and preventing the abovementioned pathologies thus forms part of the invention, as does the use of the said biotinylated heparins for the manufacture of a medicament for treating or preventing these pathologies.

According to another of its aspects, a subject of the present invention is a pharmaceutical composition comprising, as active principle, a biotinylated heparin according to the invention or a pharmaceutically acceptable salt thereof, and also at least one pharmaceutically acceptable inert excipient. The said excipients are chosen according to the desired pharmaceutical form and mode of administration, for example the oral, sublingual, subcutaneous, intramuscular, intravenous, transdermal, transmucosal, local or rectal route.

In each dosage unit, the active principle is present in the amounts suited to the envisaged daily doses in order to obtain the desired prophylactic or therapeutic effect. Each dosage unit may contain from 25 to 150 mg and advantageously from 30 to 100 mg of active principle. These anticoagulant compounds may be neutralized with avidin or streptavidin.

There may be special cases where higher or lower dosages are appropriate; such dosages are not outside the context of the invention. According to the usual practice, the dosage that is suitable for each patient is determined by the doctor according to the mode of administration and the weight and response of the said patient.

The compounds according to the invention may also be used in combination with one or more other active principles that are useful for the desired therapy, such as antithrombotic agents, anticoagulants or anti-platelet aggregating agents.

A subject of the present invention is also a process using avidin or streptavidin, characterized in that it makes it possible to neutralize the biotinylated heparins according to the invention. Thus, the avidin or streptavidin may be used for the preparation of medicaments for neutralizing the biotinylated heparins according to the present invention.

We claim:

1. A biotinylated heparin, comprising constituent polysaccharides, wherein the constituent polysaccharides have at their reducing ends a covalent bond to a group —(R1)ᵢ-Biot and correspond to the formula (I): 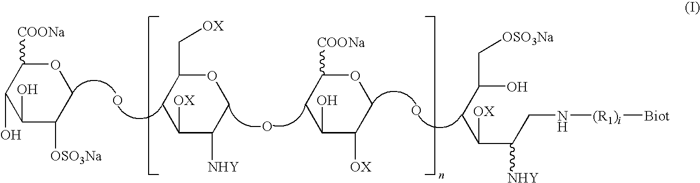
in which:
i is equal to 0 or 1,
R1 represents a sequence of formula (a) or (b): 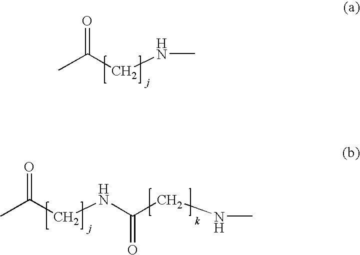
in which j and k, which may be identical or different, are integers that may take any value from 1 to 10,
Biot represents a biotin group or a biotin derivative,
n represents an integer with a mean value of about 25 for a heparin with an average molecular mass of 15 000 Da,
X represents H or SO₃Na,
Y represents COCH₃ or SO₃Na, and
the wavy line denotes a bond located either below or above the plane of the pyranose ring to which it is attached,
or the pharmaceutically acceptable salts thereof.

2. The biotinylated heparin according to claim 1, wherein i is equal to 0.

3. The biotinylated heparin according to claim 1, wherein i is equal to 1 and R1 represents a sequence of formula (a) in which j is equal to 5.

4. The biotinylated heparin according to claim 1, wherein i is equal to 1 and R1 represents a sequence of formula (b) in which j and k are identical and are equal to 5.

5. The biotinylated heparin according to claim 1, wherein Biot represents the biotin group of formula (c):

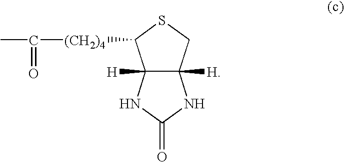

6. The biotinylated heparin according to claim 1, wherein at least 64% of the constituent polysaccharides have at their reducing ends a covalent bond to a group —(R1)ᵢ-Biot.

7. The biotinylated heparin according to claim 6, wherein at least 80% of the constituent polysaccharides have at their reducing end a covalent bond to a group —(R1)ᵢ-Biot.

8. The biotinylated heparin according to claim 1, wherein the heparin is of porcine origin.

9. The biotinylated heparin according to claim 8, wherein the heparin is derived from pig intestinal mucosae.

10. A process for preparing the biotinylated heparin as defined in claim 1, the process comprising the following steps:
a) treating heparin with heparinase 3,
b) performing a reductive amination of the treated heparin in the presence of an amine salt and a reducing agent, at a temperature of between 20 and 80° C., and
c) then performing an acylation of the product of step b) with an activated group —(R1)ᵢ-Biot, in which R1, i and Biot are as defined in claim 1, in the presence of a base in aqueous medium or in organic medium.

11. A process for preparing the biotinylated heparin as defined in claim 1, the process comprising the following steps:
a) treating heparin with heparinase 3,
b) performing a reductive amination of the treated heparin in the presence of an ammonium halide salt and a borohydride salt, at a temperature of between 50 and 80° C., and
c) then performing an acylation of the product of step b) with a group —(R1)ᵢ-Biot in activated ester form, in which R1, i and Biot are as defined in claim 1, in the presence of a base in aqueous medium.

12. A pharmaceutical composition comprising, as active principle, a biotinylated heparin according to claim 1 and at least one pharmaceutically acceptable excipient.

13. A method for the treatment or prevention of thrombosis in a patient, the method comprising administering to the patient an antithrombotic effective amount of a biotinylated heparin according to claim 1.

14. The method according to claim 13, wherein the biotinylated heparin is adminstered for treating and preventing venous thrombosis, arterial thrombotic accidents, peripheral arterial thrombosis, cerebral arterial thrombosis or strokes, the proliferation of smooth muscle cells, or angiogenesis, or as a neuroprotective agent for atherosclerosis and arteriosclerosis.

15. The method according to claim 14 wherein the arterial thrombotic accidents involve myocardial infarction or unstable angina, and the peripheral arterial thrombosis involves arteriopathy of the lower limbs.

16. A method for neutralizing the biotinylated heparin according to claim 1, the method comprising using a neutralizing amount of avidin or streptavidin.

17. The method according to claim 13 further comprising administering avidin or streptavidin to neutralize the antithrombotic effect of the biotinylated heparin.
